(19) United States
(12) Patent Application Publication
(10) Pub. No.: US 2026/0043745 A1
(43) Pub. Date: Feb. 12, 2026

(54) METHOD AND DEVICE FOR DETECTING WELDING STATE

(71) Applicant: Panasonic Intellectual Property Management Co., Ltd., Osaka (JP)

(72) Inventors: TATSURO SHIRAISHI, Osaka (JP); IZURU NAKAI, Osaka (JP); KAZUKI FUJIWARA, Osaka (JP); KOJI FUNAMI, Kyoto (JP)

(21) Appl. No.: 19/360,896

(22) Filed: Oct. 16, 2025

Related U.S. Application Data
(63) Continuation of application No. PCT/JP2024/010417, filed on Mar. 18, 2024.

(30) Foreign Application Priority Data
Apr. 25, 2023 (JP) 2023-071392

(51) Int. Cl. G01N 21/71; B23K 26/03; G01N 21/55; G01N 33/207

(52) U.S. Cl. CPC G01N 21/71; B23K 26/032; G01N 21/55; G01N 33/207

(57) ABSTRACT

A detection method of the present disclosure is a method for detecting a welding state executed by a processor, the method including acquiring a first signal intensity indicating an intensity of thermal radiation light generated from a portion irradiated with laser light and a second signal intensity indicating an intensity of reflected light reflected from the portion irradiated with the laser light, and detecting a welding state of the portion irradiated with the laser light based on the first signal intensity and the second signal intensity, in which the detecting the welding state determines whether or not the first signal intensity is less than or equal to a first reference signal intensity of thermal radiation light and the second signal intensity is greater than a second reference signal intensity of reflected light.

### TECHNICAL FIELD

The present disclosure relates to a method and a device for detecting a welding state.

### BACKGROUND ART

PTL 1 discloses a method for detecting a welding state. The detection method described in PTL 1 includes a step of detecting reflected light from a portion irradiated with laser light and light emission in the portion irradiated with laser light, and a step of detecting a welding state of the portion irradiated with laser light based on the detected reflected light and the detected light emission. In the step of detecting a welding state, it is detected whether or not a signal level of light emission is more than or equal to a predetermined first threshold value and a signal level of reflected light is less than or equal to a predetermined second threshold value.

### Citation List

### Patent Literature

PTL 1: Unexamined Japanese Patent Publication No. 2022-092729

### SUMMARY OF THE INVENTION

### Solution to problem

A method for detecting a welding state according to one aspect of the present disclosure is a method for detecting a welding state executed by a processor, the method including:

acquiring a first signal intensity indicating an intensity of thermal radiation light generated from a portion irradiated with laser light and a second signal intensity indicating an intensity of reflected light reflected from the portion irradiated with the laser light; and

detecting a welding state of the portion irradiated with the laser light based on the first signal intensity and the second signal intensity, in which

the detecting the welding state determines whether or not the first signal intensity is less than or equal to a first reference signal intensity of thermal radiation light and the second signal intensity is greater than a second reference signal intensity of reflected light.

A device for detecting a welding state according to one aspect of the present disclosure includes:

a processor; and

a storage device that stores a command executed by the processor, in which

the command includes:

acquiring a first signal intensity indicating an intensity of thermal radiation light generated from a portion irradiated with laser light and a second signal intensity indicating an intensity of reflected light reflected from the portion irradiated with the laser light; and

detecting a welding state of the portion irradiated with the laser light based on the first signal intensity and the second signal intensity, wherein

the detecting the welding state determines whether or not the first signal intensity is less than or equal to a first reference signal intensity of thermal radiation light and the second signal intensity is greater than a second reference signal intensity of reflected light.

### BRIEF DESCRIPTION OF THE DRAWINGS

FIG. 1 is a schematic diagram for explaining a concept of the present disclosure.

FIG. 2A is a schematic diagram for explaining an example of a mechanism of a recess generated by laser welding.

FIG. 2B is a schematic diagram for explaining an example of a mechanism of a recess generated by laser welding.

FIG. 2C is a schematic diagram for explaining an example of a mechanism of a recess generated by laser welding.

FIG. 3 is a schematic block diagram illustrating an example of a configuration of a laser machining system including a detection device according to a first exemplary embodiment of the present disclosure.

FIG. 4 is a schematic diagram illustrating an example of a configuration of the laser machining device.

FIG. 5 is a flowchart illustrating an example of processing of the detection device according to a first exemplary embodiment of the present disclosure.

FIG. 6 is a flowchart illustrating an example of processing of detecting a welding state.

FIG. 7 is a flowchart illustrating an example of processing of detecting a detection time domain.

FIG. 8 is a graph illustrating an example of signal intensity of thermal radiation light.

FIG. 9 is a schematic enlarged diagram in which a Z1 portion of FIG. 8 is enlarged.

FIG. 10 is a graph illustrating an example of signal intensity of reflected light.

FIG. 11 is a flowchart illustrating an example of processing of generating first reference signal intensity of thermal radiation light and determining a first threshold value.

FIG. 12 is a flowchart illustrating an example of processing of generating second reference signal intensity of reflected light and determining a second threshold value.

FIG. 13 is a flowchart illustrating another example of the processing of detecting a detection time domain.

### (BACKGROUND TO PRESENT DISCLOSURE)

In a welding apparatus that irradiates an object with laser light to perform welding, when an object is irradiated with laser light, a welding defect due to a material of the object or the like may occur. As one of the welding defects, there is a recess of a solidified portion after melting. Generation of the recess may not only impair the appearance but also cause insufficient strength of a joint portion to be joined by welding. Further, depending on degree of the recess, there is a possibility that the recess progresses into a hole.

In the method described in PTL 1, in order to detect a recess due to scattering of molten metal generated at the time of laser welding, decrease in signal level of reflected light from a portion irradiated with laser light and increase in signal level of light emission from a laser irradiator are detected. By this, in the method described in PTL 1, it is determined that a recess occurs in a portion irradiated with laser light.

However, a mechanism of generation of a recess by laser welding varies depending on a process of a welding method, a welding material, or the like. For this reason, in the method described in PTL 1, there is a case where a welding state cannot be detected depending on a welding method, a welding material, or the like. For example, in a case where a metal plate and a plurality of metal plates are welded, a welding state cannot be detected by the detection method described in PTL 1.

In view of the above, the present inventors have found a configuration capable of detecting a welding state in a case where a metal plate and a plurality of metal plates are welded, and have conceived the present disclosure.

### [Concept]

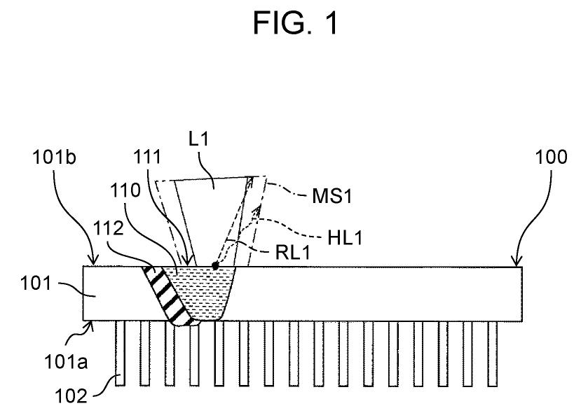

First, a concept of the present disclosure will be described with reference to FIG. 1. FIG. 1 is a schematic diagram for describing a concept of the present disclosure. FIG. 1 illustrates a state in which object 100 is laser welded.

As illustrated in FIG. 1, object 100 to be welded with laser light L1 includes first metal plate 101 and a plurality of second metal plates 102. First metal plate 101 is a plate having first surface 101a and second surface 101b facing first surface 101a. Second metal plate 102 is metal having thickness smaller than that of first metal plate 101, and is, for example, metal foil.

The plurality of second metal plates 102 are arranged on first surface 101a of first metal plate 101. The plurality of second metal plates 102 are in contact with first surface 101a of first metal plate 101.

The plurality of second metal plates 102 are arranged at intervals in a direction orthogonal to a normal direction of first surface 101a of first metal plate 101. In the present embodiment, the plurality of second metal plates 102 are arranged at equal intervals in a direction orthogonal to the normal direction of first surface 101a of first metal plate 101.

Each of the plurality of second metal plates 102 extends in a direction intersecting first surface 101a of first metal plate 101. In the present embodiment, each of the plurality of second metal plates 102 extends in a direction orthogonal to first surface 101a of first metal plate 101.

In the example illustrated in FIG. 1, in order to weld first metal plate 101 and the plurality of second metal plates 102, second surface 101b of first metal plate 101 is irradiated with laser light L1. Specifically, second surface 101b of first metal plate 101 is irradiated with laser light L1 being scanned in a direction in which the plurality of second metal plates 102 are arranged.

A portion irradiated with laser light L1 has high temperature and forms molten portion 110. Molten portion 110 is a portion where metal is in a molten state. Molten portion 110 decreases in temperature when laser light L1 is not applied and becomes solidified portion 112. Solidified portion 112 is a portion in which molten metal is solidified.

When second surface 101b of first metal plate 101 is irradiated with laser light L1, reflected light RL1 of laser light L1 is generated with respect to a direction of irradiation with laser light L1. Reflected light RL1 is laser light L1 reflected from molten surface 111 of molten portion 110. Further, thermal radiation light HL1 is also generated from molten surface 111 with respect to a direction of irradiation with laser light L1. Thermal radiation light HL1 is light radiated from molten surface 111 of molten portion 110 having high temperature due to irradiation with laser light L1.

Intensity of reflected light RL1 changes depending on a state of molten surface 111 of molten portion 110, and intensity of thermal radiation light HL1 changes depending on temperature of molten surface 111.

A method and a device for detecting a welding state of the present disclosure measure signal intensities of thermal radiation light HL1 and reflected light RL1 detected in measurement range MS1, and detect a recess generated during welding based on a change in the two signal intensities.

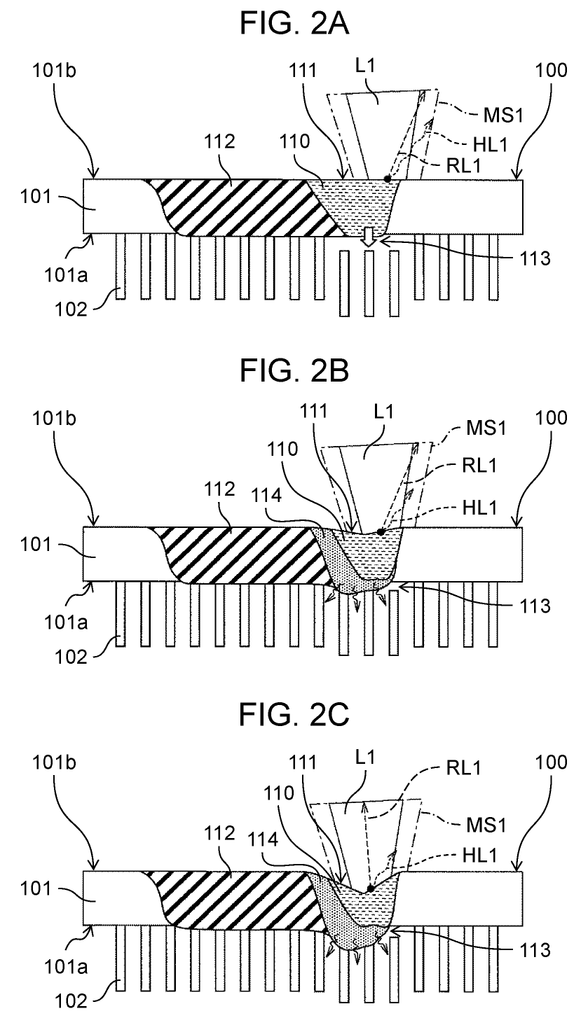

A mechanism of a recess generated during welding will be described with reference to FIGS. 2A to 2C. FIGS. 2A to 2C illustrate schematic diagrams for explaining an example of a mechanism of a recess generated by laser welding.

FIG. 2A illustrates a state in which gap 113 is formed between first metal plate 101 and the plurality of second metal plates 102. As illustrated in FIG. 2A, when gap 113 exists between first metal plate 101 and the plurality of second metal plates 102, molten portion 110 moves to a space of gap 113.

FIG. 2B shows a state in which a recess is generated in molten surface 111. As illustrated in FIG. 2B, movement of molten portion 110 to gap 113 progresses, and a recess starts to be generated in molten surface 111 of molten portion 110. Further, when movement of molten portion 110 to gap 113 progresses and molten portion 110 comes into contact with second metal plate 102, heat of molten portion 110 moves to second metal plate 102 due to a difference in thermal conductivity between molten portion 110 and second metal plate 102. By this, temperature of molten portion 110 decreases, and cooled portion 114 is formed. Cooled portion 104 is a portion where temperature decreases in molten portion 110. By this, intensity of thermal radiation light HL1 generated from molten surface 111 decreases.

FIG. 2C illustrates a state where the recess of molten portion 110 progresses. As illustrated in FIG. 2C, when the recess becomes large in molten portion 110, inclination of molten surface 111 changes, and a reflection direction of reflected light RL1 changes. Specifically, reflected light RL1 is reflected in a direction substantially parallel to an irradiation direction of laser light L1. That is, when a scale of a recess becomes large in molten portion 110, specularly reflected light returns to a portion where reflected light RL1 is detected. As a result, intensity of reflected light RL1 detected in measurement range MS1 increases.

From the above, in welding between first metal plate 101 and the plurality of second metal plates 102, a welding state can be detected based on decrease in intensity of thermal radiation light HL1 and increase in intensity of reflected light RL1.

Hereinafter, one exemplary embodiment of the present disclosure will be described with reference to the accompanying drawings. Note that, description below is merely exemplary in nature, and is not intended to limit the present disclosure, its application, or its use. Moreover, the drawings are schematic representations, and a ratio between dimensions or the like do not necessarily match an actual one.

### (First exemplary embodiment)

### [Overall configuration]

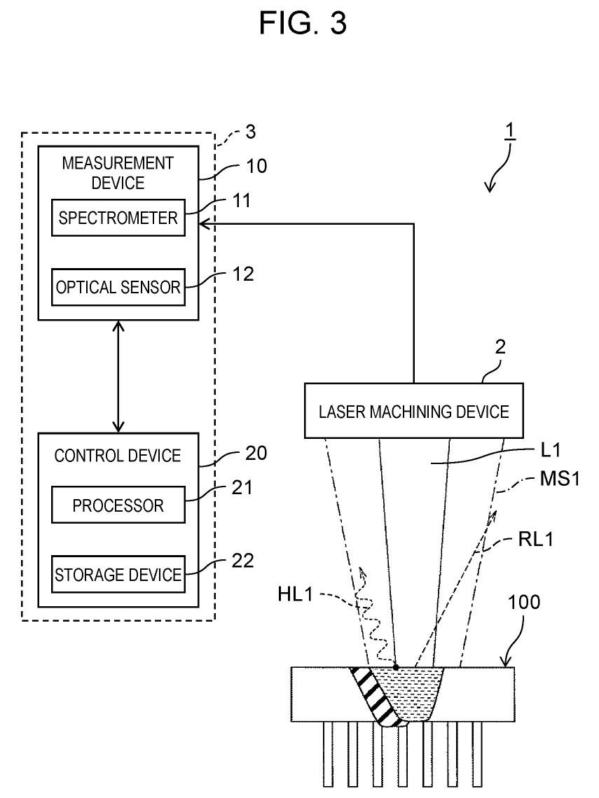

FIG. 3 is a schematic block diagram illustrating an example of a configuration of laser machining system 1 including detection device 3 according to a first exemplary embodiment of the present disclosure.

As illustrated in FIG. 3, laser machining system 1 includes laser machining device 2 and detection device 3.

### <Laser machining device>

Laser machining device 2 irradiates object 100 with laser light L1 to perform laser welding. Laser machining device 2 is arranged above object 100 at a predetermined distance. The predetermined distance is set such that a spot diameter of laser light L1 on a surface of object 100 has appropriate size for welding. Further, laser machining device 2 guides thermal radiation light HL1 and reflected light RL1 generated in a portion irradiated with laser light L1 in object 100 to detection device 3.

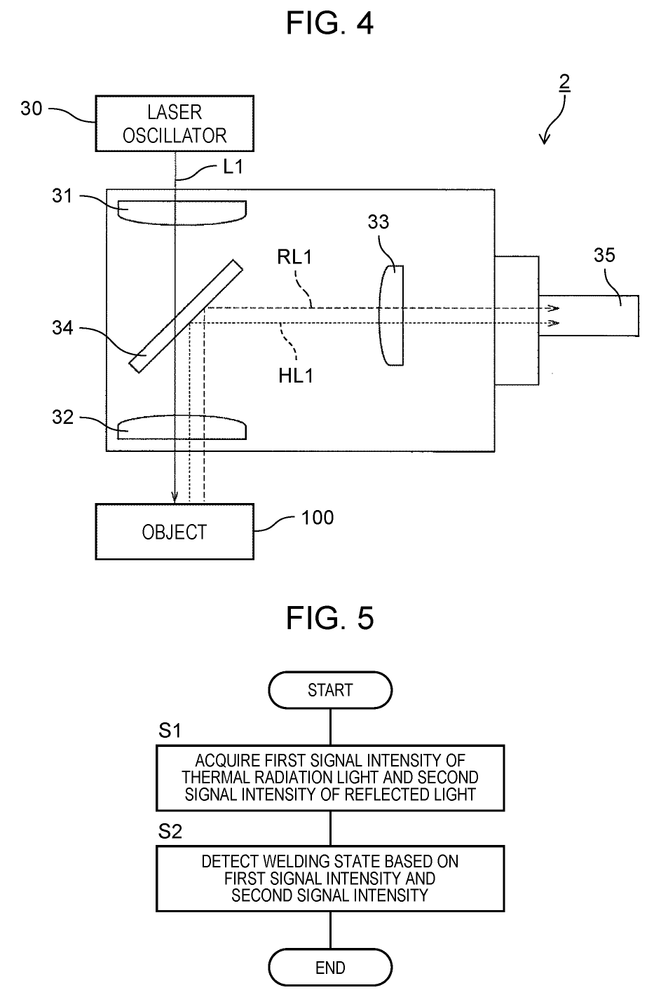

FIG. 4 is a schematic diagram illustrating an example of a configuration of laser machining device 2. As illustrated in FIG. 4, laser machining device 2 includes laser oscillator 30, lenses 31 to 33, half mirror 34, and optical fiber 35.

Laser oscillator 30 outputs laser light L1. Laser light L1 output from laser oscillator 30 is collimated by lens 31. Collimated laser light L1 enters lens 32 through half mirror 34. Laser light L1 is focused by lens 32 and applied to object 100.

Thermal radiation light HL1 and reflected light RL1 are generated from a portion irradiated with laser light L1 in object 100. Thermal radiation light HL1 and reflected light RL1 are received by lens 32. Optical axes of thermal radiation light HL1 and reflected light RL1 received by lens 32 are converted by, for example, 90° by half mirror 34 and thermal radiation light HL1 and reflected light RL1 are incident on lens 33. Thermal radiation light HL1 and reflected light RL1 are focused on optical fiber 35 by lens 33. Optical fiber 35 transmits thermal radiation light HL1 and reflected light RL1 to detection device 3.

Here, measurement range MS1 is determined by a core diameter of optical fiber 35 and focal length of the lenses 32 and 33.

Note that the configuration of laser machining device 2 described above is an example, and is not limited to the present disclosure. For example, laser machining device 2 may include a galvanometer mirror arranged between half mirror 34 and lens 32. Laser light L1 may be scanned on object 100 by a galvanometer mirror.

### <Detection device>

Returning to FIG. 3, detection device 3 includes measurement device 10 and control device 20.

Measurement device 10 measures first signal intensity indicating intensity of thermal radiation light HL1 generated from a portion irradiated with laser light L1 and second signal intensity indicating intensity of reflected light RL1 reflected from a portion irradiated with laser light L1.

For example, measurement device 10 includes spectrometer 11 and optical sensor 12.

Spectrometer 11 disperses light transmitted from optical fiber 35 into thermal radiation light HL1 and reflected light RL1. For example, spectrometer 11 separates light by a wavelength of light. For example, a wavelength of thermal radiation light HL1 is 1300 nm, and a wavelength of reflected light RL1 is 515 nm. Spectrometer 11 includes, for example, a half mirror, a diffraction grating, and the like.

Optical sensor 12 detects the first signal intensity of thermal radiation light HL1 and the second signal intensity of reflected light RL1 dispersed by spectrometer 11.

Measurement device 10 includes two optical sensors 12 for receiving thermal radiation light HL1 and reflected light RL1. The two optical sensors are sensitive to wavelengths of thermal radiation light HL1 and reflected light RL1. For example, optical sensor 12 is an element that outputs voltage when light is input. For example, optical sensor 12 may be a photodiode or the like.

The first signal intensity of thermal radiation light HL1 and the second signal intensity of reflected light RL1 detected by optical sensor 12 are transmitted to control device 20.

Note that the configuration of measurement device 10 described above is an example, and is not limited to the present disclosure.

Control device 20 controls measurement device 10. Control device 20 receives the first signal intensity of thermal radiation light HL1 and the second signal intensity of reflected light RL1 from measurement device 10, and determines a welding state based on the first signal intensity and the second signal.

Control device 20 includes processor 21 and storage device 22. Control device 20 realizes a predetermined function by processor 21 executing a command stored in storage device 22. A function of control device 20 may be configured only with hardware or may be realized by a combination of hardware and software. Control device 20 may include one or more processors 21.

Processor 21 can include, for example, a microcomputer, a CPU, an MPU, a GPU, a DSU, an FPGA, an ASIC, and the like. Processor 21 may be configured with a dedicated electronic circuit designed to realize a predetermined function.

Storage device 22 is a storage medium that stores a program and data for realizing a function of control device 20. Storage device 22 can be realized by a hard disk (HDD), an SSD, a RAM, a DRAM, a ferroelectric memory, a flash memory, a magnetic disk, or a combination of these, for example.

For example, control device 20 converts a voltage signal acquired from measurement device 10 into a digital signal by AD change, and performs signal processing as waveform data. The waveform data to which the signal processing is performed is stored in storage device 22.

### [Operation]

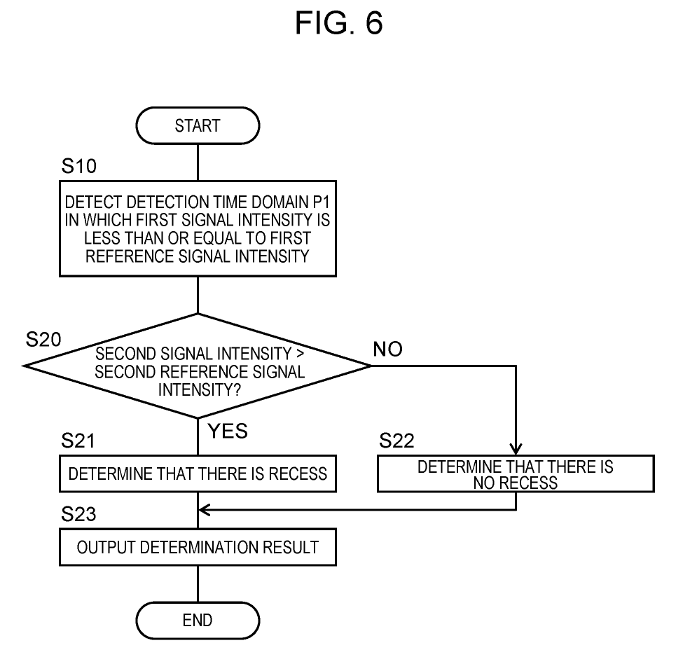

Next, an example of operation of detection device 3, that is, an example of a method for detecting a welding state will be described with reference to FIGS. 5 and 6.

FIG. 5 is a flowchart illustrating an example of processing of detection device 3 according to the first exemplary embodiment of the present disclosure. FIG. 6 is a flowchart illustrating an example of processing of detecting a welding state.

As illustrated in FIG. 5, detection device 3 performs steps S1 and S2.

In step S1, detection device 3 acquires the first signal intensity indicating intensity of thermal radiation light HL1 and the second signal intensity indicating intensity of reflected light RL1.

In the present embodiment, spectrometer 11 disperses light transmitted from optical fiber 35 into thermal radiation light HL1 and reflected light RL1. Optical sensor 12 receives dispersed thermal radiation light HL1 and reflected light RL1, and detects the first signal intensity of thermal radiation light HL1 and the second signal intensity of reflected light RL1. The detected first signal intensity and second signal intensity are transmitted to control device 20. By this, control device 20 acquires the first signal intensity and the second signal intensity.

In step S2, detection device 3 detects a welding state based on the first signal intensity of thermal radiation light HL1 and the second signal intensity of reflected light RL1.

For example, in step S2, steps S10 and S20 to S23 illustrated in FIG. 6 are performed.

As illustrated in FIG. 6, in step S10, detection time domain P1 in which the first signal intensity of thermal radiation light HL1 is less than or equal to first reference signal intensity is detected.

The first reference signal intensity is signal intensity serving as a reference of thermal radiation light HL1 generated from molten surface 111 during laser welding, and is signal intensity of thermal radiation light HL1 in a normal welding state without an abnormality such as a recess. For example, the first reference signal intensity is signal intensity of an average waveform of thermal radiation light HL1 in a normal welding state.

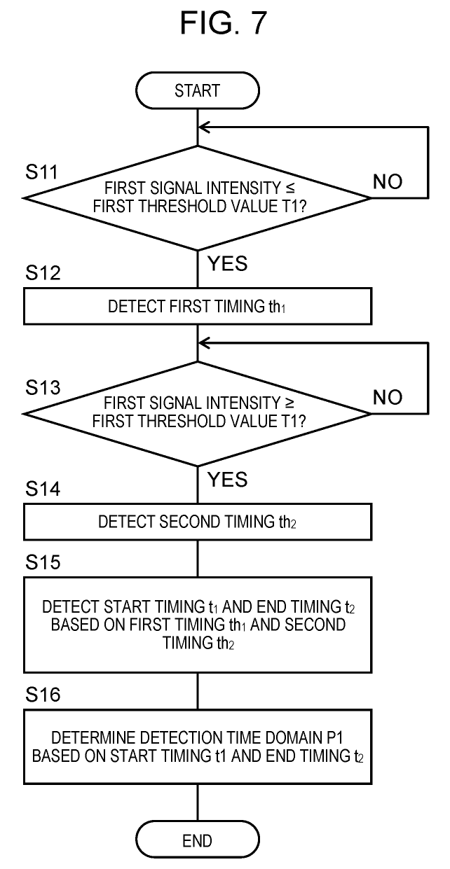

In the present embodiment, in step S10, detection time domain P1 is detected using first threshold value T1 smaller than the first reference signal intensity. Hereinafter, step S10 will be described in detail with reference to FIGS. 7 to 9.

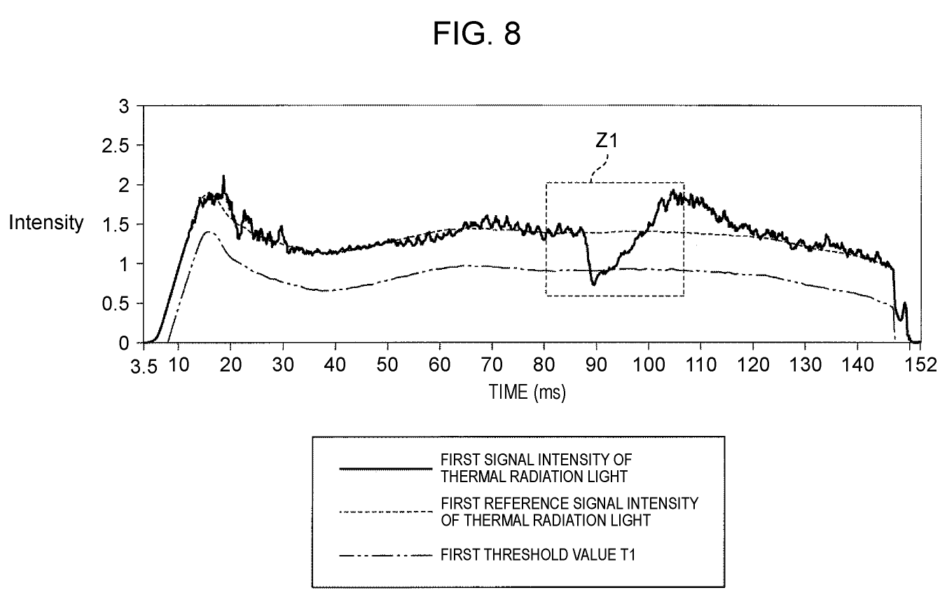

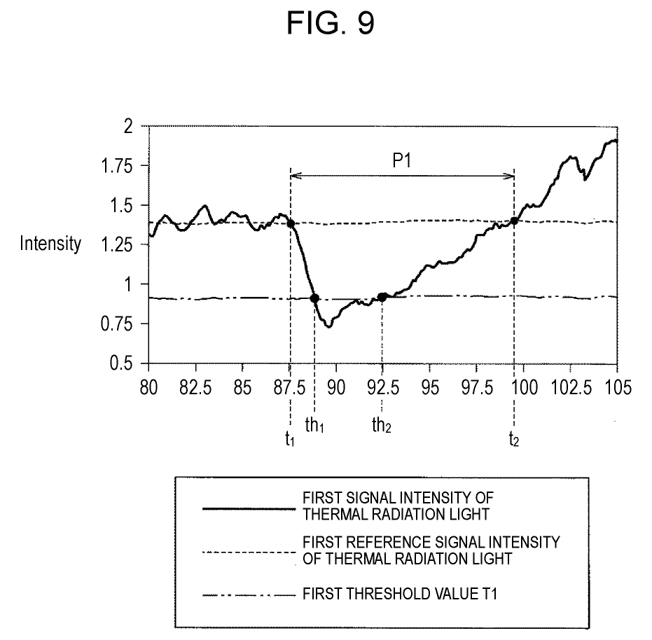

FIG. 7 is a flowchart illustrating an example of processing of detecting detection time domain P1. FIG. 8 is a graph illustrating an example of the first signal intensity of thermal radiation light HL1. FIG. 9 is a schematic enlarged diagram in which a Z1 portion of FIG. 8 is enlarged. Note that the graphs illustrated in FIGS. 8 and 9 illustrate the first signal intensity, the first reference signal intensity, and first threshold value T1 of thermal radiation light HL1 in a case where a recess is generated.

In the example illustrated in FIG. 7, steps S11 to S16 are performed in processing of detecting detection time domain P1.

In step S11, detection device 3 determines whether or not the first signal intensity is less than or equal to first threshold value T1 smaller than the first reference signal intensity.

The first reference signal intensity and/or first threshold value T1 are stored in storage device 22, for example.

As illustrated in FIGS. 8 and 9, in a case where no recess is generated in molten portion 110, signal intensity of thermal radiation light HL1 does not decrease as indicated by the first reference signal intensity. On the other hand, in a case where a recess is generated in molten portion 110, thermal radiation light HL1 becomes smaller than the first reference signal intensity as indicated by the first signal intensity.

As described above, detection device 3 determines whether or not the first signal intensity becomes less than or equal to first threshold value T1 smaller than the first reference signal intensity. When determining that the first signal intensity is greater than first threshold value T1, detection device 3 repeats step S11. When determining that the first signal intensity is less than or equal to first threshold value T1, detection device 3 performs step S12.

In step S12, detection device 3 detects first timing th₁ at which the first signal intensity becomes less than or equal to first threshold value T1.

In step S13, detection device 3 determines whether or not the first signal intensity is more than or equal to first threshold value T1 after first timing th₁. In a case of determining that the first signal intensity is smaller than first threshold value T1, detection device 3 repeats step S13. In a case of determining that the first signal intensity is more than or equal to first threshold value T1, detection device 3 performs step S14.

In step S14, detection device 3 detects second timing th₂ at which the first signal intensity becomes more than or equal to first threshold value T1 after first timing th₁.

In step S15, detection device 3 detects start timing t₁ at which the first signal intensity starts to become smaller than the first reference signal intensity and end timing t₂ at which the first signal intensity becomes greater than the first reference signal intensity based on first timing th₁ and second timing th₂.

For example, before first timing th₁, detection device 3 detects start timing t₁ at which the first signal intensity decreases to less than or equal to the first reference signal intensity. For example, there may be a plurality of timings at which the first signal intensity becomes less than or equal to the first reference signal intensity. Among them, start timing t₁ is a timing before first timing th₁ and closest to first timing th₁.

For example, detection device 3 detects end timing t₂ at which the first signal intensity becomes more than or equal to the first reference signal intensity after second timing th₂. For example, there may be a plurality of timings at which the first signal intensity becomes more than or equal to the first reference signal intensity. Among them, end timing t₂ is a timing after second timing th₂ and closest to second timing th₂.

In step S16, detection device 3 determines detection time domain P1 based on start timing t1 and end timing t2.

As described above, in step S10, detection time domain P1 is detected by performing steps S11 to S16.

Returning to FIG. 6, in step S20, detection device 3 determines whether or not the second signal intensity of reflected light RL1 is greater than the second reference signal intensity in detection time domain P1.

The second reference signal intensity is signal intensity serving as a reference of reflected light RL1 generated from molten surface 111 during laser welding, and is signal intensity of reflected light RL1 in a normal welding state without an abnormality such as a recess. For example, the second reference signal intensity is signal intensity of an average waveform of reflected light RL1 in a normal welding state.

In a case of determining that the second signal intensity of reflected light RL1 is greater than the second reference signal intensity in detection time domain P1 in step S20, detection device 3 proceeds to step S21 and determines that “there is a recess”. In a case of determining that the second signal intensity is less than or equal to the second reference signal intensity in detection time domain P1, detection device 3 proceeds to step S22 and determines that “there is no recess”.

In step S23, detection device 3 outputs a determination result. For example, detection device 3 may output a flag indicating whether or not there is a recess. Alternatively, detection device 3 may output information on whether or not there is a recess in an output device such as a display.

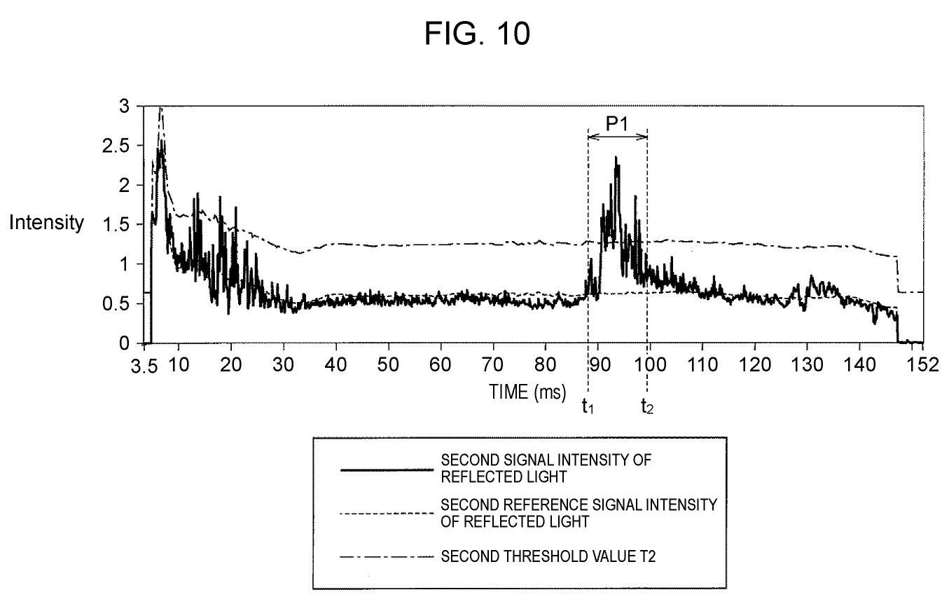

In the present embodiment, in step S20, detection device 3 determines whether or not there is a recess by using second threshold value T2 greater than the second reference signal intensity. Hereinafter, step S20 will be described in detail with reference to FIG. 10.

FIG. 10 is a graph illustrating an example of signal intensity of reflected light RL1. Note that the graph illustrated in FIG. 10 illustrates the second signal intensity, the second reference signal intensity, and the second threshold value of reflected light RL1 in a case where a recess is generated.

In the present embodiment, as illustrated in FIG. 10, detection device 3 determines whether or not the second signal intensity of reflected light RL1 is more than or equal to second threshold value T2 greater than the second reference signal intensity in detection time domain P1.

The second reference signal intensity and/or second threshold value T2 are stored in storage device 22, for example.

In a case of determining that the second signal intensity is more than or equal to second threshold value T2 in detection time domain P1, detection device 3 determines that “there is a recess”. In a case of determining that the second signal intensity is smaller than second threshold value T2 in detection time domain P1, detection device 3 determines that “there is no recess”.

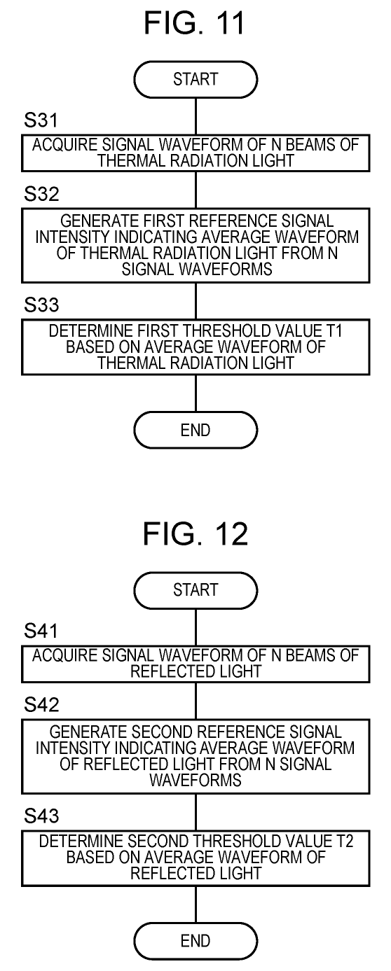

Next, an example of processing of generating the first reference signal intensity and determining first threshold value T1 will be described with reference to FIG. 11. FIG. 11 is a flowchart illustrating an example of processing of generating the first reference signal intensity of thermal radiation light HL1 and determining first threshold value T1.

As illustrated in FIG. 11, detection device 3 generates the first reference signal intensity by performing steps S31 to S33, and determines first threshold value T1.

In step S31, detection device 3 acquires signal waveforms of N beams of thermal radiation light HL1. Specifically, detection device 3 acquires signal waveforms of N beams of thermal radiation light HL1 in a normal welding state. The number N is, for example, more than or equal to ten.

In step S32, detection device 3 generates the first reference signal intensity indicating an average waveform of thermal radiation light HL1 from N signal waveforms. Specifically, detection device 3 generates an average waveform of thermal radiation light HL1 by calculating an average of N signal intensities.

In step S33, detection device 3 determines first threshold value T1 based on an average waveform of thermal radiation light HL1. For example, detection device 3 calculates a standard deviation of an average waveform of thermal radiation light HL1 and determines a lower limit value of the standard deviation as first threshold value T1. Alternatively, detection device 3 may determine a lower limit value obtained by multiplying a standard deviation of thermal radiation light HL1 by k as first threshold value T1. The number k is an integer between 1 and 5 (inclusive). Preferably, k is 5.

Next, an example of calculation of second threshold value T2 will be described with reference to FIG. 12. FIG. 12 is a flowchart illustrating an example of processing of determining second threshold value T2 of signal intensity of reflected light RL1.

As illustrated in FIG. 12, detection device 3 determines second threshold value T2 by performing steps S41 to S43.

In step S41, detection device 3 acquires signal waveforms of N beams of reflected light RL1. Specifically, detection device 3 acquires signal waveforms of N beams of reflected light RL1 in a normal welding state.

In step S42, detection device 3 generates the second reference signal intensity indicating an average waveform of reflected light RL1 from N signal waveforms. Specifically, detection device 3 generates an average waveform of reflected light RL1 by calculating an average of N signal intensities.

In step S43, detection device 3 determines second threshold value T2 based on an average waveform of reflected light RL1. For example, detection device 3 calculates a standard deviation of an average waveform of reflected light RL1, and determines an upper limit value of the standard deviation as second threshold value T2. Alternatively, detection device 3 may determine an upper limit value obtained by multiplying a standard deviation of reflected light RL1 by m as second threshold value T2. The number m is an integer between 1 and 5 (inclusive). Preferably, m is 5.

Note that the first reference signal intensity, the second reference signal intensity, first threshold value T1, and second threshold value T2 described above are not limited to the present disclosure. For example, one reference signal intensity and the second reference signal intensity may be median values of N signal waveforms. Any constants may be set as first threshold value T1 and second threshold value T2. Alternatively, first threshold value T1 and second threshold value T2 may be determined based on a maximum value of N signal waveforms.

Further, in a case where it is determined that there is no recess in detection of a welding state by detection device 3, detection device 3 may use the first signal intensity and the second signal intensity used for the detection when determining the first reference signal intensity, the second reference signal intensity, first threshold value T1, and second threshold value T2 described above. By this, the first reference signal intensity, the second reference signal intensity, first threshold value T1, and second threshold value T2 can be updated.

### [Effect]

According to the first exemplary embodiment, the method for detecting a welding state includes step S1 of acquiring signal intensity of thermal radiation light HL1 and reflected light RL1, and step S2 of detecting a welding state. In step S1, first signal intensity indicating intensity of thermal radiation light HL1 generated from a portion irradiated with laser light L1 and second signal intensity indicating intensity of reflected light RL1 reflected from a portion irradiated with laser light L1 are acquired. In step S2, a welding state of a portion irradiated with laser light L1 is detected based on the first signal intensity and the second signal intensity. Further, in step S2, it is determined whether or not the first signal intensity is less than or equal to the first reference signal intensity of thermal radiation light HL1 and the second signal intensity is greater than the second reference signal intensity of reflected light RL1. With such a configuration, a welding state can be detected in a case where a metal plate and a plurality of metal plates are welded. For example, in a case where first metal plate 101 having a predetermined surface and the plurality of second metal plates 102 arranged along a direction orthogonal to a normal direction of the predetermined surface are welded, a welding state such as a recess of a portion irradiated with laser light L1 can be detected.

Step S2 of detecting a welding state includes step S11 of detecting detection time domain P1 and step S12 of determining whether or not the second signal intensity is greater than the second reference signal intensity in detection time domain P1. Detection time domain P1 is a time domain in which the first signal intensity is less than or equal to the first reference signal intensity. With such a configuration, it is possible to identify a detection region of the second signal intensity of reflected light RL1 from detection time domain P1 where the first signal intensity of thermal radiation light HL1 decreases. As a result, a welding state can be efficiently detected.

Step S11 of detecting detection time domain P1 includes steps S21 to S24 of detecting first timing th₁ and second timing th₂, step S25 of detecting start timing t₁ and end timing t₂, and step S26 of determining detection time domain P1. First timing th₁ is a timing at which the first signal intensity becomes less than or equal to first threshold value T1 smaller than one reference signal intensity, and second timing th₂ is a timing at which the first signal intensity becomes more than or equal to first threshold value T1 after first timing th₁. Start timing t₁ is a timing at which the first signal intensity starts to become smaller than the first reference signal intensity, and end timing t₂ is a timing at which the first signal intensity becomes greater than the first reference signal intensity. Start timing t₁ and end timing t₂ are detected based on first timing th₁and second timing th₂. Detection time domain P1 is determined based on start timing t₁ and end timing t₂. With such a configuration, a welding state can be detected with high accuracy.

Step S2 of detecting a welding state includes determining whether or not the second signal intensity is more than or equal to second threshold value T2 greater than the second reference signal intensity. With such a configuration, a welding state can be detected with higher accuracy.

The first reference signal intensity is signal intensity of an average waveform of thermal radiation light HL1 in a normal welding state, and the second reference signal intensity is signal intensity of an average waveform of reflected light RL1 in a normal welding state. With such a configuration, a welding state can be detected with high accuracy.

The first reference signal intensity is signal intensity of an average waveform of thermal radiation light HL1 in a normal welding state, and first threshold value T1 is determined by a lower limit value obtained by multiplying a standard deviation of the average waveform of thermal radiation light HL1 by k. The number k is an integer between 1 and 5 (inclusive). With such a configuration, detection time domain P1 can be accurately detected.

The second reference signal intensity is signal intensity of an average waveform of reflected light RL1 in a normal welding state, and second threshold value T2 is determined by an upper limit value obtained by multiplying a standard deviation of the average waveform of reflected light RL1 by m. The number m is an integer between 1 and 5 (inclusive). With such a configuration, a welding state can be detected with higher accuracy.

In step S2 of detecting a welding state, in a case where it is determined that the first signal intensity is less than or equal to the first reference signal intensity and the second signal intensity is greater than the second reference signal intensity, it is determined that there is a recess. With such a configuration, it is possible to determine a recess of a portion irradiated with laser light L1 and to detect a welding abnormality.

Note that detection device 3 also achieves a similar effect as the detection method described above.

In the present exemplary embodiment, the example in which detection device 3 includes measurement device 10 is described, but the present invention is not limited to this. For example, detection device 3 does not need to include measurement device 10. Detection device 3 only needs to include a processor and storage device 22 that stores a command executed by the processor.

For example, information on the first signal intensity and the second signal intensity detected by measurement device 10 may be stored in a server, and detection device 3 may acquire the first signal intensity and the second signal intensity from the server via a wired or wireless network.

In the present embodiment, the example in which detection of detection time domain P1 is performed using first threshold value T1 is described, but the present invention is not limited to this. The detection of detection time domain P1 may be performed without using first threshold value T1 as long as a time domain in which the first signal intensity of thermal radiation light HL1 is less than or equal to the first reference signal intensity can be detected.

In the present embodiment, the example in which a recess is detected using second threshold value T2 is described, but the present invention is not limited to this. The detection of a recess may be performed without using second threshold value T2 as long as determination can be made based on the fact that the second signal intensity of reflected light RL1 is greater than the second reference signal intensity.

### (First variation)

In a first variation, another example of the processing of detecting detection time domain P1 will be described.

In the first variation, detection time domain P1 is detected in a case where a state in which the first signal intensity of thermal radiation light HL1 is less than or equal to the first reference signal intensity continues for predetermined time or longer.

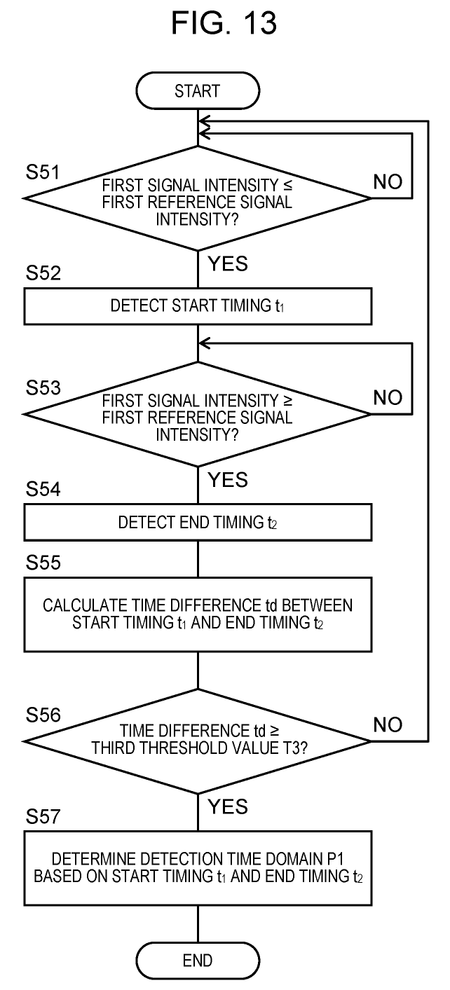

FIG. 13 is a flowchart illustrating another example of the processing of detecting detection time domain P1. As illustrated in FIG. 13, in the first variation, detection time domain P1 is detected as steps S51 to S57 are performed.

In step S51, detection device 3 determines whether or not the first signal intensity of thermal radiation light HL1 is less than or equal to the first reference signal intensity. In a case where the first signal intensity of thermal radiation light HL1 is less than or equal to the first reference signal intensity, detection device 3 performs step S52. In a case where the first signal intensity of thermal radiation light HL1 is greater than the first reference signal intensity, detection device 3 repeats step S51.

In step S52, detection device 3 detects start timing t₁ at which the first signal intensity of thermal radiation light HL1 starts to decrease to less than or equal to the first reference signal intensity.

In step S53, detection device 3 determines whether or not the first signal intensity of thermal radiation light HL1 is more than or equal to the first reference signal intensity after start timing t₁. In a case where the first signal intensity of thermal radiation light HL1 is more than or equal to the first reference signal intensity, detection device 3 performs step S54. In a case where the first signal intensity of thermal radiation light HL1 is smaller than the first reference signal intensity, detection device 3 repeats step S53.

In step S54, detection device 3 detects end timing t₂ at which the first signal intensity becomes more than or equal to the first reference signal intensity after start timing t₁.

In step S55, detection device 3 calculates time difference td between start timing t₁ and end timing t₂. Time difference td is time from start timing t₁ to end timing t₂.

In step S56, detection device 3 determines whether or not time difference td is more than or equal to third threshold value T3. Third threshold value T3 may be an arbitrary constant. For example, third threshold value T3 is set to more than or equal to 5 ms. In a case where time difference td is more than or equal to third threshold value T3, detection device 3 performs step S57. In a case where time difference td is smaller than third threshold value T3, detection device 3 returns to step S51.

In step S57, detection device 3 determines detection time domain P1 based on start timing t₁ and end timing t₂.

As described above, in the first variation, detection time domain P1 is determined as steps S51 to S57 are performed.

Note that, in the first variation, the example in which detection time domain P1 is determined based on determination as to whether or not time difference td is more than or equal to third threshold value T3 is described, but the present invention is not limited to this. For example, in a case where predetermined time or more elapses from start timing t₁ in a state where the first signal intensity of thermal radiation light HL1 is less than or equal to the first reference signal intensity, detection device 3 may detect end timing t₂and determine detection time domain P1.

The exemplary embodiment is described above to exemplify the technique disclosed in the present application. However, the technique according to the present disclosure is not limited to the above, and is applicable to an exemplary embodiment in which a change, replacement, addition, omission, or the like is made as appropriate.

Further, the terms “first”, “second”, and the like used herein are only for the purpose of description, and should not be understood as explicitly or implicitly indicating relative importance or a priority of technical features. Features limited to “first” and “second” are intended to explicitly or implicitly indicate inclusion of one or more of the features.

Although the present disclosure is fully described with reference to a preferred exemplary embodiment and with reference to the accompanying drawings, various variations and modifications are clear to those skilled in the art. Such variations and modifications are to be understood as being included within the scope of the present disclosure as set forth in the appended claims, unless departing from the scope of the present disclosure.

Further, a general and specific aspect of the preset disclosure may be realized by a system, a method, a computer program, a computer-readable recording medium, and a combination of these.

### (Overview of exemplary embodiment)

(1) A detection method of one aspect of the present disclosure is a method for detecting a welding state executed by a processor, the method including acquiring a first signal intensity indicating an intensity of thermal radiation light generated from a portion irradiated with laser light and a second signal intensity indicating an intensity of reflected light reflected from the portion irradiated with the laser light, and detecting a welding state of the portion irradiated with the laser light based on the first signal intensity and the second signal intensity, in which the detecting the welding state determines whether or not the first signal intensity is less than or equal to a first reference signal intensity of thermal radiation light and the second signal intensity is greater than a second reference signal intensity of reflected light.

(2) In the detection method of (1), the detecting the welding state may include detecting a detection time domain in which the first signal intensity is less than or equal to the first reference signal intensity, and determining whether or not the second signal intensity is greater than the second reference signal intensity in the detection time domain.

(3) In the detection method of (2), the detecting the detection time domain may include detecting a first timing at which the first signal intensity becomes less than or equal to a first threshold value smaller than the first reference signal intensity and a second timing at which the first signal intensity becomes greater than or equal to the first threshold value after the first timing, detecting a start timing at which the first signal intensity starts to become smaller than the first reference signal intensity and an end timing at which the first signal intensity becomes greater than the first reference signal intensity based on the first timing and the second timing, and determining the detection time domain from the start timing and the end timing.

(4) In the detection method of (2), the detecting the detection time domain includes detecting the detection time domain in a case where a state in which the first signal intensity is less than or equal to the first reference signal intensity continues for predetermined time or more.

(5) In the detection method of any one of (1) to (4), the detecting the welding state may include determining whether or not the second signal intensity is greater than or equal to a second threshold value that is greater than the second reference signal intensity.

(6) In the detection method of any one of (1) to (5), the first reference signal intensity may be a signal intensity of an average waveform of thermal radiation light in a normal welding state, and the second reference signal intensity may be a signal intensity of an average waveform of reflected light in a normal welding state.

(7) In the detection method of (3), the first reference signal intensity may be a signal intensity of an average waveform of thermal radiation light in a normal welding state, the first threshold value may be determined by a lower limit value obtained by multiplying a standard deviation of an average waveform of the thermal radiation light by k, and k may be an integer between 1 and 5, inclusive.

(8) In the detection method of (5), the second reference signal intensity may be a signal intensity of an average waveform of reflected light in a normal welding state, the second threshold value may be determined by an upper limit value obtained by multiplying a standard deviation of an average waveform of the reflected light by m, and m may be an integer between 1 and 5, inclusive.

(9) In the detection method of any one of (1) to (8), the detecting the welding state may determine that a recess is present in a case where the first signal intensity is determined to be less than or equal to the first reference signal intensity and the second signal intensity is determined to be greater than the second reference signal intensity.

(10) A detection device according to one aspect of the present disclosure includes a processor, and a storage device that stores a command executed by the processor, in which the command includes acquiring a first signal intensity indicating an intensity of thermal radiation light generated from a portion irradiated with laser light and a second signal intensity an indicating intensity of reflected light reflected from the portion irradiated with the laser light, and detecting a welding state of a portion irradiated with the laser light based on the first signal intensity and the second signal intensity, in which the detecting the welding state determines whether or not the first signal intensity is less than or equal to a first reference signal intensity of thermal radiation light and the second signal intensity is greater than a second reference signal intensity of reflected light.

(11) In the detection device of (10), the detecting the welding state may include detecting a detection time domain in which the first signal intensity is less than or equal to the first reference signal intensity, and determining whether or not the second signal intensity is greater than the second reference signal intensity in the detection time domain.

(12) In the detection device of (11), the detecting the detection time domain may include detecting a first timing at which the first signal intensity becomes less than or equal to a first threshold value smaller than the first reference signal intensity and a second timing at which the first signal intensity becomes greater than or equal to the first threshold value after the first timing, detecting a start timing at which the first signal intensity starts to become smaller than the first reference signal intensity and an end timing at which the first signal intensity becomes greater than the first reference signal intensity based on the first timing and the second timing, and determining the detection time domain from the start timing and the end timing.

(13) In the detection device of (11), the detecting the detection time domain includes detecting the detection time domain in a case where a state in which the first signal intensity is less than or equal to the first reference signal intensity continues for predetermined time or more.

(14) In the detection device of any one of (10) to (13), the detecting the welding state may include determining whether or not the second signal intensity is greater than or equal to a second threshold value that is greater than the second reference signal intensity.

(15) In the detection device of any one of (10) to (14), the first reference signal intensity may be a signal intensity of an average waveform of thermal radiation light in a normal welding state, and the second reference signal intensity may be a signal intensity of an average waveform of reflected light in a normal welding state.

(16) In the detection device of (12), the first reference signal intensity may be a signal intensity of an average waveform of thermal radiation light in a normal welding state, the first threshold value may be determined by multiplying a standard deviation of an average waveform of the thermal radiation light by k, and k may be an integer between 1 and 5, inclusive.

(17) In the detection device of (14), the second reference signal intensity may be a signal intensity of an average waveform of reflected light in a normal welding state, the second threshold value may be determined by multiplying a standard deviation of an average waveform of the reflected light by m, and m may be an integer between 1 and 5, inclusive.

(18) In the detection device of any one of (10) to (17), the detecting the welding state may determine that a recess is present in a case where the first signal intensity is determined to be less than or equal to the first reference signal intensity and the second signal intensity is determined to be greater than the second reference signal intensity.

(19) A program according to one aspect of the present disclosure causes a processor to execute the method of any one of (1) to (9).

(20) A non-transitory computer readable storage medium according to an aspect of the present disclosure stores a program for causing a processor to execute the method of any one of (1) to (9).

According to the present disclosure, it is possible to provide a detection method and a detection device capable of detecting a welding state in a case where a metal plate and a plurality of metal plates are welded.

### INDUSTRIAL APPLICABILITY

The present disclosure can be applied to a device and a method for detecting a welding state in welding using laser light.

### REFERENCE MARKS IN THE DRAWINGS

1 laser machining system

2 laser machining device

3 detection device

10 measurement device

11 spectrometer

12 optical sensor

20 control device

21 processor

22 storage device

30 laser oscillator

31 lens

32 lens

33 lens

34 half mirror

35 optical fiber

100 object

101 first metal plate

101a first surface

101b second surface

102 second metal plate

110 molten portion

111 molten surface

112 solidified portion

113 gap

114 cooled portion

L1 laser

HL1 thermal radiation light

RL1 reflected light

MS1 measurement range

P1 detection time domain

T1 first threshold value

T2 second threshold value

T3 third threshold value

1. A method for detecting a welding state executed by a processor, the method comprising:
acquiring a first signal intensity indicating an intensity of thermal radiation light generated from a portion irradiated with laser light and a second signal intensity indicating an intensity of reflected light reflected from the portion irradiated with the laser light; and
detecting a welding state of the portion irradiated with the laser light based on the first signal intensity and the second signal intensity, wherein
the detecting the welding state determines whether or not the first signal intensity is less than or equal to a first reference signal intensity of thermal radiation light and the second signal intensity is greater than a second reference signal intensity of reflected light.

2. The method according to claim 1, wherein
the detecting the welding state includes:
detecting a detection time domain in which the first signal intensity is less than or equal to the first reference signal intensity; and
determining whether or not the second signal intensity is greater than the second reference signal intensity in the detection time domain.

3. The method according to claim 2, wherein
the detecting the detection time domain includes:
detecting a first timing at which the first signal intensity becomes less than or equal to a first threshold value smaller than the first reference signal intensity and a second timing at which the first signal intensity becomes greater than or equal to the first threshold value after the first timing;
detecting a start timing at which the first signal intensity starts to become smaller than the first reference signal intensity and an end timing at which the first signal intensity becomes greater than the first reference signal intensity based on the first timing and the second timing; and
determining the detection time domain from the start timing and the end timing.

4. The method according to claim 2, wherein the detecting the detection time domain includes detecting the detection time domain in a case where a state in which the first signal intensity is less than or equal to the first reference signal intensity continues for predetermined time or more.

5. The method according to claim 1, wherein the detecting the welding state includes determining whether or not the second signal intensity is greater than or equal to a second threshold value that is greater than the second reference signal intensity.

6. The method according to claim 1, wherein
the first reference signal intensity is a signal intensity of an average waveform of thermal radiation light in a normal welding state, and
the second reference signal intensity is a signal intensity of an average waveform of reflected light in a normal welding state.

7. The method according to claim 3, wherein
the first reference signal intensity is a signal intensity of an average waveform of thermal radiation light in a normal welding state,
the first threshold value is determined by a lower limit value obtained by multiplying a standard deviation of an average waveform of the thermal radiation light by k, and
k is an integer between 1 and 5, inclusive.

8. The method according to claim 5, wherein
the second reference signal intensity is a signal intensity of an average waveform of reflected light in a normal welding state,
the second threshold value is determined by an upper limit value obtained by multiplying a standard deviation of an average waveform of the reflected light by m, and
m is an integer between 1 and 5, inclusive.

9. The method according to claim 1, wherein the detecting the welding state determines that a recess is present in a case where the first signal intensity is determined to be less than or equal to the first reference signal intensity and the second signal intensity is determined to be greater than the second reference signal intensity.

10. A device for detecting a welding state, the device comprising:
a processor; and
a storage device that stores a command executed by the processor, wherein
the command includes:
acquiring a first signal intensity indicating an intensity of thermal radiation light generated from a portion irradiated with laser light and a second signal intensity indicating an intensity of reflected light reflected from the portion irradiated with the laser light; and
detecting a welding state of the portion irradiated with the laser light based on the first signal intensity and the second signal intensity, wherein
the detecting the welding state determines whether or not the first signal intensity is less than or equal to a first reference signal intensity of thermal radiation light and the second signal intensity is greater than a second reference signal intensity of reflected light.

11. The device according to claim 10, wherein
the detecting the welding state includes:
detecting a detection time domain in which the first signal intensity is less than or equal to the first reference signal intensity; and
determining whether or not the second signal intensity is greater than the second reference signal intensity in the detection time domain.

12. The device according to claim 11, wherein
the detecting the detection time domain includes:
detecting a first timing at which the first signal intensity becomes less than or equal to a first threshold value smaller than the first reference signal intensity and a second timing at which the first signal intensity becomes greater than or equal to the first threshold value after the first timing;
detecting a start timing at which the first signal intensity starts to become smaller than the first reference signal intensity and an end timing at which the first signal intensity becomes greater than the first reference signal intensity based on the first timing and the second timing; and
determining the detection time domain from the start timing and the end timing.

13. The device according to claim 11, wherein the detecting the detection time domain includes detecting the detection time domain in a case where a state in which the first signal intensity is less than or equal to the first reference signal intensity continues for predetermined time or more.

14. The device according to claim 10, wherein the detecting the welding state includes determining whether or not the second signal intensity is greater than or equal to a second threshold value that is greater than the second reference signal intensity.

15. The device according to claim 10, wherein
the first reference signal intensity is a signal intensity of an average waveform of thermal radiation light in a normal welding state, and
the second reference signal intensity is a signal intensity of an average waveform of reflected light in a normal welding state.

16. The device according to claim 12, wherein
the first reference signal intensity is a signal intensity of an average waveform of thermal radiation light in a normal welding state,
the first threshold value is determined by multiplying a standard deviation of an average waveform of the thermal radiation light by k, and
k is an integer between 1-5., inclusive.

17. The device according to claim 14, wherein
the second reference signal intensity is a signal intensity of an average waveform of thermal radiation light in a normal welding state, and
the second threshold value is determined by multiplying a standard deviation of an average waveform of the reflected light by m, and
m is an integer between 1-5., inclusive.

18. The device according to claim 10, wherein the detecting the welding state determines that a recess is present in a case where the first signal intensity is determined to be less than or equal to the first reference signal intensity and the second signal intensity is determined to be greater than the second reference signal intensity.

19. A non-transitory computer readable storage medium storing a program for causing a processor to execute the method according to claim 1.
